(19) United States
(12) Patent Application Publication
(10) Pub. No.: US 2017/0281737 A1
(43) Pub. Date: Oct. 5, 2017

(54) UNIVERSAL PLATFORM FOR TARGETING THERAPIES TO TREAT NEUROLOGICAL DISEASES

(71) Applicants: THE MEDICAL COLLEGE OF WISCONSIN, INC., Milwaukee, WI (US); WISCONSIN ALUMNI RESEARCH FOUNDATION, Madison, WI (US)

(72) Inventors: Joseph T. Barbieri, New Berlin, WI (US); Chen Chen, Brookfield, WI (US); Amanda Przedpelski, Franklin, WI (US); Eric A. Johnson, Madison, WI (US); Sabine Pellett, Madison, WI (US)

(21) Appl. No.: 15/509,371

(22) PCT Filed: Sep. 29, 2015
(86) PCT No.: PCT/US15/52886

§ 371 Date: Mar. 7, 2017

Related U.S. Application Data
(60) Provisional application No. 62/057,447, filed on Sep. 30, 2014.

(51) Int. Cl. A61K 38/50; C07K 14/245; C12N 9/86; C07K 16/12

(52) U.S. Cl. CPC A61K 38/50; C12Y 305/02006; C07K 16/1282; C07K 14/245; C12N 9/86; C07K 2317/76; C07K 2317/22; C07K 2317/622; C07K 2317/92

(57) ABSTRACT

The present invention provides a universal delivery platform of functional, heterologous compounds to specific cells using toxins modified to include a heterologous compound. In one embodiment, the toxin is an AB5 toxin. In one embodiment, the AB5 toxin is a heat-labile enterotoxin from E. coli (LT), including LTI, LTII, LTIIa, LTIIb, LTIIc and other recombinant forms of LT. Methods of use are also provided.

### CROSS-REFERENCE TO RELATED APPLICATIONS

This application claims the benefit of U.S. Provisional Patent Application No. 62/057,447, filed Sep. 30, 2014; which is incorporated herein by reference as if set forth in its entirety.

### STATEMENT REGARDING FEDERALLY SPONSORED RESEARCH OR DEVELOPMENT

This invention was made with government support under AI101313 and AI057153 awarded by the National Institutes of Health. The government has certain rights in the invention.

### BACKGROUND OF THE INVENTION

Conventionally, there are two approaches to delivering heterologous proteins into cells; viral-based and protein-based. Protein-based therapies lack a genetic, infectious component, an advantage over viral-based therapies. For instance, the Protective Antigen (PA) of anthrax toxin has been developed as a heterologous protein delivery system. The PA delivery platform is efficient and ubiquitous, since the anthrax toxins receptors are common among cell types. However, this is also a limiting feature of PA as a cell-type specific protein delivery platform. Immunotoxins (IT) are another platform for heterologous protein delivery. IT specificity is enhanced by identifying receptors that have elevated expression of a host receptor on a targeted cancer cell relative to “normal” cells. However, this limits the type of cells that can be targeted. Accordingly, there is a need for a delivery platform that can deliver functional therapies into specific cells, such as neurons.

Botulism is a rare and potentially fatal paralytic illness caused by botulinum neurotoxins (BoNTs), with an estimated human median lethal dose (LD-50) of 1-2 ng/kg intravenously or intramuscularly and 10-20 ng/kg when inhaled. BoNTs enter neurons of the peripheral nervous system and target and cleave soluble NSF attachment protein receptors (SNARE) proteins for a prolonged time (up to 6 months), which causes flaccid paralysis and can lead to death in severe cases. There are no vaccines or therapies against botulism. While naturally occurring botulism is rare (about 150 cases per year), the threat of natural or intentional outbreaks exist, and would require prolonged, supportive therapy. Since the extended paralysis of botulism is due to prolonged activity of intracellular BoNT, development of a therapy platform for BoNT intoxication should target neurons and directly inactivate intracellular toxin. Although significant progress has been made for BoNT vaccines and neutralizing antibodies, specific therapies targeting intracellular BoNTs has not been developed to deliver specific compounds capable of inhibiting intracellular BoNT.

Protein aggregation in the brain is a characteristic feature of neurodegenerative disorders such as Alzheimer's disease (AD), Parkinson's disease (PD), amyotrophic lateral sclerosis (ALS) and Huntington's disease (HD). Targeted therapies that slow to progression of these neurodegenerative disorders, versus merely mitigating the symptoms of the disease, are also not available at this time.

Gangliosides, glycosphingolipids that contain sialic acids, are components of all animal cell membranes and are particularly abundant in the plasma membranes of neurons. The brain contains as much as 20 to 500 times more gangliosides than most non-neural tissues. In the nervous system of higher vertebrates, complex gangliosides such as GM1a, GD1a, GD1b and GT1b comprise about 80-90% of the total gangliosides, whereas the simple gangliosides GM2 and GM3 are more commonly displayed on extra-neuronal tissue and are largely absent on brain tissue. Platforms recognizing gangliosides, in particular complex gangliosides, therefore hold great promise for delivery of therapeutics to neurons.

AB5 toxins are synthesized by several bacterial pathogens and plants, comprising a monomeric enzymatic A subunit and pentameric binding B subunit. The A subunit is a single polypeptide composed of two domains, A1 and A2, which are linked together via a disulfide bond. The A1 domain includes the catalytic domain responsible for toxicity to the host cell. The A2 domain consists of an α-helix that penetrates into the central pore of the B-subunit, thereby non-covalently anchoring the A-subunit and B-subunits together to create the holotoxin.

There are four main families of the AB5 toxin: the cholera toxin (CT) family, Pertussis toxin, Shiga toxin, and subtilase cytotoxin. The cholera toxin (CT) family includes CT from Vibrio cholerae as well as the heat-labile enterotoxins (LT) of Escherichia coli: LTI, LTIIa, LTIIb and LTIIc. LT is a multimeric protein composed of two functionally distinct domains: an enzymatically active A subunit having ADP-ribosylating activity, and a pentameric B subunit that contains GM1 (momosialoganglioside) receptor-binding site. Table 1 shows the biochemical and biological properties of the CT and LT AB5 toxins. The CT- and LT-like toxins enter host cells by binding gangliosides on the cell surface, leading to endocytosis. During the past two decades, CT and LT have been deployed as adjuvants that stimulate immunity, or alternatively, suppression of autoimmunity. CT and LT utilize gangliosides as host receptors. For example, CT, LTI and LTIIc bind GM1a; LTIIa binds GD1b; and LTIIb binds GD1a.

TABLE 1
AB5 toxins.

| | | Host | Catalytic | Host | A subunit | B subunit |
|---|---|---|---|---|---|---|
| Toxin | Source | Receptor | activity | Target | (aa) | (aa) |
| CT | V. cholerae | GM1a | ADP-r | Gₛ | 240 | 103 |
| LTI | E. coli | GM1a | ADP-r | Gₛ | 240 | 103 |
| LTIIa | E. coli | GD1b | ADP-r | Gₛ | 237 | 100 |
| LTIIb | E. coli | GD1a | ADP-r | Gₛ | 237 | 99 |
| LTIIc | E. coli | GM1a | ADP-r | Gₛ | 241 | 98 |

```
Gal-GalNA c-Gal-Glc-Cer Gal-GalNAc-Gal-Glc-Cer Gal-GalNAc-Gal-Glc-Cer Gal-GalNAc-Gal-Glc-Cer
|  |  |  |  |  |
SA SA GT1b SA GD1b SA SA GD1a SA GM1a
                        |  |
                        SA SA
CT = cholera toxin; LT = heat-liable enterotoxin of Escherichia coli
```

There remains is a need for therapeutics that can at least slow the progression of neurodegenerative disease versus merely mitigating symptoms of neurodegenerative disease.

### BRIEF SUMMARY OF THE INVENTION

Provided herein is a universal delivery platform of functional, heterologous compounds to specific cells using toxins modified to include a heterologous compound. In one embodiment, the toxin is an AB5 toxin. In one embodiment, the AB5 toxin is a heat-labile enterotoxin from E. coli (LT).

In a first aspect, provided herein is a delivery platform comprising a toxin modified to incorporate a heterologous compound. The toxin can be an AB5 toxin. The toxin can be modified to remove the A1 domain. In some cases, the toxin is selected from the group consisting to CT, LTI, LTIIa, LTIIb, LTIIC, and any recombinant LT derivative. The toxin can be LTIIa and the heterologous compound can be β-lactamase. However, in other embodiments, any toxin effectively transferring a functional heterologous compound may be used. Any heterologous compound may be delivered using the present invention. In some cases, the heterologous compound is selected from the group consisting of β-lactamase, camelid antibodies, Adam10 and related proteases, TFEB, E3 binding protein, BDNF, protease, kinase, P53, PTEN, pRb, SOD1, HFE, NOD2, CARD15, P53, PTEN, pRB, and GMCSF.

In another aspect, provided herein is a method of treating botulism comprising administering a delivery platform comprising an AB5 toxin modified to incorporate β-lactamase to a subject in need thereof, where the botulism is treated. The AB5 toxin can be LTIIa.

In a further aspect, provided herein is a method of treating Parkinson's Disease (PD) where the method comprising administering a delivery platform comprising a toxin modified to incorporate a heterologous compound to a subject in need thereof, where the heterologous compound comprises a neuroprotective agent, and where the PD is treated. In some cases, the toxin is an AB5 toxin. The toxin can be modified to remove the A1 domain. In some cases, the AB5 toxin is LTIIa. The neuroprotective agent can be selected from the group consisting of BDNF, Brn4, and Progranulin.

In another aspect, provided herein is a method of treating Cystic Fibrosis (CF) comprising administering a delivery platform comprising a toxin modified to incorporate a heterologous compound to a subject in need thereof, where the heterologous compound comprises a CFTR polypeptide, and where the CF is treated. In some cases, the toxin is an AB5 toxin. The toxin can be modified to remove the A1 domain. In some cases, the AB5 toxin is LTIIa.

The invention also provides a method of treating Parkinson's Disease comprising administering a delivery platform comprising an AB5 toxin modified to incorporate BDNF to a subject in need thereof, wherein the Parkinson's Disease is treated. In one embodiment, the AB5 toxin is LTIIa.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows expression of LTIIa and βlac-LTIIa. (Upper) The genes encoding the A and B subunit of LTIIa were expressed in pET28, using a di-cistronic T7 promoter. The B subunit was modified to contain HA-His6 epitopes for immune detection and purification, respectively. The gene encoding β-lactamase was subcloned to replace the A1 subunit (βlac-LTIIa). (Middle) Schematic of AB5 organization of LTIIa, βlac-LTIIa, and βlacₙᵤₗₗ-LTIIa. (Lower) Purified LTIIa, βlac-LTIIa, and βlacₙᵤₗₗ-LTIIa separated by SDS-PAGE and stained by Coomassie blue. Ratio of B subunit to A subunit was determined by densitometry (B/A ratio is listed below each lane).

FIGS. 2A-2B show dose-dependent delivery of βlac by βlac-LTIIa. Rat primary cortical neurons were incubated with 40 nM βlac-LTIIa at 37° C. for 60 min. Cells were incubated with CCF2-AM at RT for 30 min followed by IF to detect LTIIa bound (anti-HA, left-hand column) and βlac (anti-FLAG, second from left). Uncleaved CCF2 is shown (second from right), and cleaved CCF2 (CCF2C) is shown in cyan (right-hand column). (B) Rat primary cortical neurons were incubated with 40 nM LTIIa or 0.1 to 40 nM βlac-LTIIa at 37° C. for 60 min. Cleavage of CCF2 was quantified using the ratio of fluorescence of cleaved CCF2 to that of CCF2 as a function of added βlac-LTIIa.

FIGS. 3A-3C show that LTIIa delivers cargo (βlac) more efficiently into GD1b enriched Neuro-2a cells relative to GM1a enriched Neuro-2a cells. Neuro-2a cells were loaded with 10 μg/ml of ganglioside GD1b or GM1a in DMEM with 0.5% FBS at 37° C. for 3 h. Cells were washed and incubated with 40 nM of βlac-LTIIa_{B} or LTIIa at 37° C. for 60 min. Cells were loaded with CCF2AM at RT for 30 min followed by IF staining using anti-HA antibody (red). Uncleaved CCF2 was shown in green and cleaved CCF2 (CCF2c) was shown in cyan. (B) Cleavage of substrate CCF2 was quantified using the ratio of fluorescent intensities CCF2c/CCF2/HA. (C) Neuro-2a cells were loaded with GD1b and then incubated with 40 nM βlac-LTIIa, βlacₙᵤₗₗ-LTIIa or LTIIa at 37° C. for 60 min alone or with 0.1 μg/ml of brefeldin A (BFA). Cells were washed and were loaded with CCF2AM at RT for 30 min. Cleavage of CCF2 was quantified using the ratio of fluorescent intensities CCF2c /CCF2/HA.

FIGS. 4A-4B show delivery and separation of β-lac from the B subunit during β-lac-LTIIa entry into neurons. Rat primary cortical neurons were incubated with 40 nM βlac_{F}-LTIIa at 4° C. or 37° C. for 60 min. Cells were washed, followed by IF staining, using anti-HA antibody (green) and anti-FLAG antibody (red). (B) Representative colocalization between HA and FLAG staining were shown by cytofluogram with Pearson's coefficient (PC) determined.

FIGS. 5A-5C demonstrate that βlac-LTIIa cleaves CCF2 in BoNT-intoxicated primary cortical neurons. Rat cortical primary neurons were incubated with 2 nM of BoNT/D at 37° C. for 16 h. BoNT-treated neurons were incubated with 40 nM β-lac-LTIIa at 37° C. for 60 min alone or with 0.1 μg/ml brefeldin A (BFA), washed and were loaded with CCF2AM at RT for 30 min followed by IF staining, using anti-HA antibody (red) and anti-VAMP2 (magenta) which only recognizes full-length VAMP2. Cytosolic CCF2 was shown in green and cleaved CCF2 (CCF2_{C}) was shown in cyan. (B) Cleavage of VAMP2 was quantified using the ratio of fluorescent intensities VAMP2/HA. (C) Cleavage of substrate CCF2 was quantified using the ratio of fluorescent intensities CCF2_{C}/CCF2/HA.

FIGS. 6A-6E are schematics of VHH-B8-LTIIa. A 1 nM concentration of BoNT/A was incubated with rat cortical neurons at 37° C. for 2 h, when toxin was removed and the indicated amount of VHH-B8-LTIIa (B8) was added to neurons for an additional 3 h. Cells were fixed and incubated with Alexa 647-wheat germ agglutinin (WGA; magenta) for 30 min as a cell marker. The IF assay stained for HA (red) and cleaved SNAP25 (SNAP25c, green) in BoNT/A-treated cells. (C) A 1 nM concentration of BoNT/D was incubated with rat cortical neurons at 37° C. for 2 h, when toxin was removed and the indicated amount of VHH-B8-LTIIa (B8) was added to neurons for an additional 3 h. Cells were fixed and incubated with Alexa 647-wheat germ agglutinin (WGA; magenta) for 30 min as a cell marker. The IF assay stained for HA (red) and cleaved SNAP25 (SNAP25c, green) in intact VAMP2 (green) in BoNT/D-treated cells. (D) Cleavage was quantified by measuring the SNAP25c/WGA ratio of fluorescence intensities for BoNT/A-treated cells and the VAMP2/WGA ratio of fluorescence intensities for BoNT/D-treated cells. (E) BoNT/A or BoNT/D (1 nM) was incubated with rat cortical neurons at 37° C. for 2 h, when toxin was removed and the indicated amount of VHH-B8-LTIIa (B8) was added with neurons overnight. Cleavage was quantified as described for panel D. Data were analyzed as SEM by two-tailed Student's t test. *, P<0.05; **, P<0.005. Bar, 20 μm. NS, not significant; o/n, overnight.

FIG. 7 demonstrates that LTIIa delivers cargo (βlac) more efficiently into GD1b-enriched Neuro-2a cells than GM1a-enriched Neuro-2a cells. Neuro-2a cells were loaded with 10 μg/ml of ganglioside GD1b or GM1a in DMEM with 0.5% FBS at 37° C. for 3 h. Cells were washed and incubated with 40 nM βlac-LTIIa or LTIIa at 37° C. for 60 min. Cells were loaded with CCF2-AM at RT for 30 min, followed by IF staining using anti-HA antibody (red). Uncleaved CCF2 is shown in green, and cleaved CCF2 (CCF2_{C}) is shown in cyan. Data were analyzed by two-tailed Student's t test. *, P<0.05; **, P<0.005; ***, P<0.001. Bar, 20 μm.

FIG. 8 shows that LTIIa delivers βlac more efficiently to neurons than to Neuro-2a cells and HeLa cells. A 40 nM concentration of βlac-LTIIa was incubated with rat cortical neurons; Neuro-2a cells loaded with GD1b or GM1a; and HeLa cells loaded with GD1b, GM1a, GM2, or GD2 at 37° C. for 60 min. Cells were loaded with CCF2-AM at RT for 30 min followed by IF staining using anti-HA antibody. Cleavage of substrate CCF2 was quantified using the CCF2c/CCF2/HA ratio of fluorescent intensities. The dashed line was drawn based on detectable translocation by IF. Data were analyzed by two-tailed Student's t test. *, P<0.05; **, P<0.005; ***, P<0.001.

FIGS. 9A-9B demonstrate engineering AB5 toxins as platforms to deliver functional heterologous proteins into neurons. AB5 toxins are composed of a catalytic domain that encodes a catalytic activity (A1), and ADP-riboxyltransferase activity for cholera toxin and heat-labile enterotoxins of E. coli, and a linker (A2). A1 and A2 are joined by a disulfide bond. A2 inserts, by noncovalent interactions, with the B5 oligomer. (B) LTIIa was constructed where the A1 domain was replaced with βlactamase (βlac-LTIIa) or a single chain camelid antibody against the LC of BoNT/A (VHH-LTIIa) which allows the measurement of protein translocation into neurons by LTIIA and inactivates LC in BoNT intoxicated neurons.

FIGS. 10A-10B. Assay of the intracellular localization of β-lactamase (upper) CCF2-AM (Invitrogen) passes across cell membranes. (Middle) Within the cytosol, CCF2-AM has an Ex 409 nm and FRET Em 520 nm (GREEN), β-lactamase cleaves CCF2 to shift Em to 447 nm (BLUE). (Lower) Schematic of the CCF2 cleavage reaction. (B). Cleavage of substrate CCF2 was quantified using the ratio of fluorescent intensities CCF_{C}/CCF2/DsRed (β-lac expression).

FIG. 11. Complex gangliosides. Shown are 4 common gangliosides of the brain. Several complex gangliosides enriched in the brain and motor neurons, including GT1b, GD1b, and GD1a, while GM1a is present in membranes of neurons and non-neuronal sources. A series gangliosides have sia6 sialicacids(SA) and b-series gangliosides have sia6 sia7 SA.

FIG. 12. Structure-based sequence alignment for CT and LTs. The ganglioside-binding regions in the CT-GM1a structure are indicated: Gal4-binding regions (cyan) and contact residues (#), Sia6-binding (green). Sia7-binding in LTIIb (mustard) and Sia5 (purple) are marked. Residues that make H bonds with the sugar residues are marked with red #. Conserved residues in all five proteins are highlighted with yellow and those conserved in only the three LTII derivatives are in pink.

FIG. 13. Engineering pan-BoNT therapies. Two approaches will encode anti-BoNT LC therapies. First, non-hydrolysable SNARE binding derivatives of SNAP25 and VAMP2 substrates SNAP25(141-206) and VAMP2(lo-94) will be engineered to encode nonhydrolysable P1′ mutations to the 7 BoNT serotypes. Initially individual high affinity (+) SNAP25 and high affinity(+) VAMP2 derivatives will replace the A1 domain of LTIIa. Next, the two SNARE therapies will be fused for intracellular delivery. Then, αLC inhibitors comprising the VHH domains of αLC/A and α-LC/B nanobodies will be delivered into the cytosol to neutralize intracellular LC activity. Initially individual camelids will be engineered will replace the A1 domain of LTIIa and then fused for intracellular delivery. To enhance potency an F Box domain will be added to target the SNARE or αLC complex for E3 ubiquitin ligase degradation.

### DETAILED DESCRIPTION OF THE INVENTION

In General. Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The systems and methods provided herein are based, at least in part, on the inventors' discovery that LT enterotoxins can deliver with specificity a therapeutic cargo to neurons to neutralize intracellular BoNT light chain activity. Accordingly, provided herein is a universal delivery platform for delivering functional, heterologous compounds to specific cell types using toxins modified for targeted delivery of functional “cargo” such as therapeutic agents and other heterologous compounds. Thus, provided herein is a new approach for targeted delivery of therapeutics for the treatment of BoNT intoxication, neurodegenerative diseases, and latent virus infection in neurons.

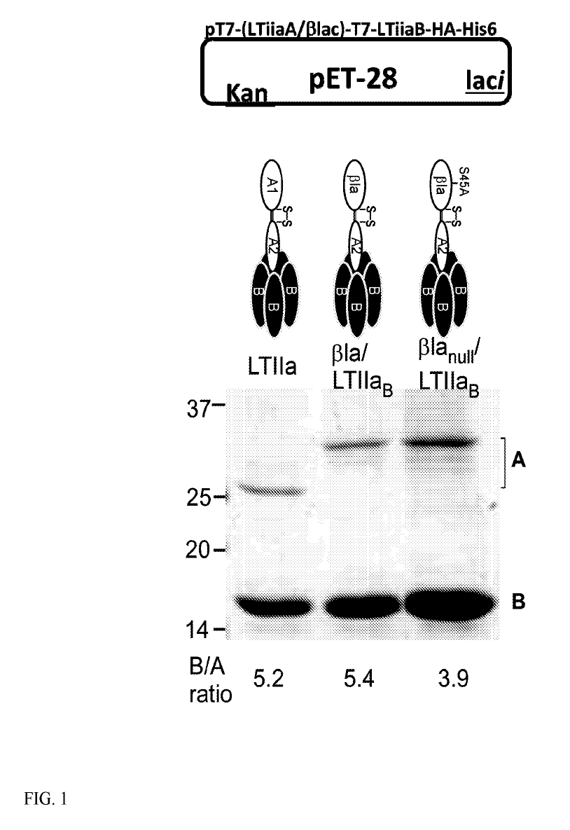

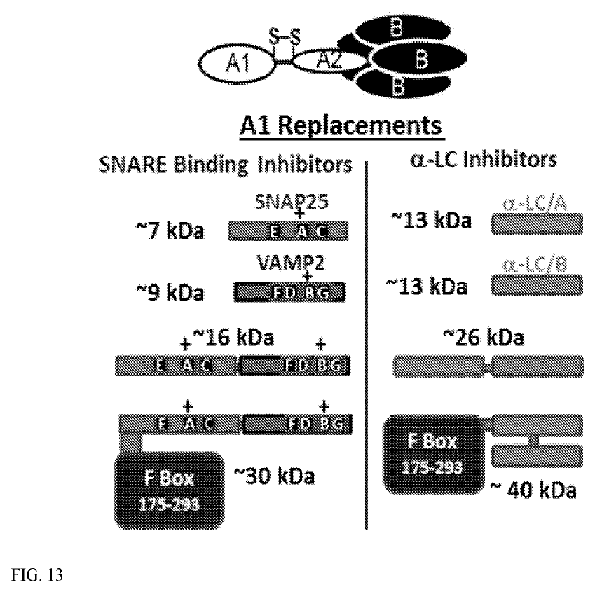

In exemplary embodiments, the universal delivery platform for delivering functional cargo comprises a toxin modified for use according to the invention is an Escherichia coli (E. coli) heat-labile enterotoxin (LT). LT is functionally, structurally, and immunologically related to cholera toxin (CT) of Vibrio cholerae (Clements et al., 1978, Infect. Immun. 21:1036-1039). LT and CT are synthesized as holotoxin molecules composed of five identical subunits B and an enzymatically active subunit A. Upon thiol reduction, subunit A dissociates into two polypeptide chains: an enzymatically active A1 peptide and a smaller A2 peptide. As used herein, the term “LT” refers to any heat-labile enterotoxin produced by any enterotoxigenic E. coli strain. The term “LT” encompasses the LTI, LTIIa, LTIIb, and LTIIc enterotoxins of E. coli as well as recombinant forms of LTI, LTIIa, LTIIb, LTIIc, where any part of the A1 subunit/domain has been replaced with a heterologous compound, including protein, carbohydrate, and/or nucleic acid. The compound would comprise a specific therapy to specific human, animal, or plant diseases. By “modified” we mean that a portion of the toxin has been replaced with the heterologous compound. In exemplary embodiments, the toxin is the heat-labile enterotoxin, AB5. In such cases, “modified” means that the toxic catalytic domain of AB5 toxin is replaced with a functional heterologous compound for delivery of functional “cargo” into the cytosol of neuronal cells (FIG. 1). For example, in some embodiments, the A1 domain of the LTIIa toxin (residues 1-172) is replaced with β-lactamase or a camelid single chain antibody that targets the Light Chain of Botulinum toxin serotype A (VHH-B8) to provide an engineered delivery platform of bacterial toxins that can deliver therapeutic compounds like β-lactamase or VHH-B8 to neurons across the blood-brain barrier. However, any area of the A domain of toxin can be effectively replaced with a specific “cargo” compound. In some cases, the B domain can be modified for delivery to specific cells, as determined by one of skill in the art (see also FIG. 13).

As used herein, the term “heterologous compound” refers to any molecule or compound different from the delivery compound. In exemplary embodiments, any molecule or compound that can provide therapeutic or functional benefit to the targeted cell can be delivered into the cell using the universal delivery platform of the present invention. Heterologous compounds include, without limitation, β-lactamase (β-lac), a camelid antibody, a zinc-dependent protease, transcription factor EB (TFEB), an E3 binding protein, brain-derived neurotrophic factor (BDNF), a protease, a kinase, p53, phosphatase and tensin homolog (PTEN), Superoxide Dismutase 1 (SOD1), human hemochromatosis protein (HFE), caspase recruitment domain-containing protein 15 (CARD15), retinoblastoma gene product (pRB), and granulocyte-macrophage colony-stimulating factor (GM-CSF).

In some cases, the modified toxin delivery platform comprises β-lac-LTIIa, where the LTIIa platform efficiently and specifically delivers cargo (e.g., a heterologous molecule such as a protein or polynucleotide) into neurons. The heterologous molecule can be covalently linked to the modified enterotoxin for targeted delivery.

By way of example, a heterologous compound or “cargo” for delivery using a platform provided herein is a neuroprotective agent modified for delivery to specific cells to treat Parkinson's disease (PD). PD is a neurodegenerative disease that may be caused by the degeneration of dopaminergic neurons. As used herein, the phrase “neuroprotective activity” refers to prevention of neural cell death. The effect may take the form of protection of neuronal cells i.e., neurons, from apoptosis or degeneration. Assays for qualifying a neuroprotective activity include cell viability assays (e.g., XTT, MTT), morphological assays (e.g., cell staining) or apoptosis biochemical assays (e.g., caspase 3 activity and the like). Compounds such as BDNF, Brn4, and Progranulin may provide a neuroprotective effect for PD or for one or more other neurodegenerative diseases. Accordingly, delivery of a neuroprotective agent (e.g., BDNF, Brn4, Progranulin) according to the delivery platform provided herein can slow clinical progression and treat symptoms of Parkinson's disease in a Parkinson's disease patient. BDNF has been known as a therapeutic agent for treatment of neurodegenerative diseases (e.g., ALS) or diabetic peripheral neuropathy (Mizisin et al., Journal of Neuropathology and Experimental Neurology 56:1290 (1997)). Brn4 is a member of the POU domain family of transcription factors. Brn4 induced the differentiation of NSCs into neurons, and co-transfection of tyrosine hydroxylase (TH) and Brn4 promotes differentiation of NSCs to mature dopamine-synthesizing neurons. PRGN is widely distributed throughout the central nervous system, acts as a regulator of neuroinflammation, and is important for long-term neuronal survival. As used herein, “Parkinson's Disease symptoms” include the commonly observed symptoms of Parkinson's Disease, such as bradykinesia, or slowness in voluntary movement, delayed transmission of signals from the brain to the skeletal muscles, tremors in the hands, fingers, forearm, foot, mouth, and chin; rigidity, and poor balance. In addition, the progressive loss of voluntary and involuntary muscle control produces a number of secondary symptoms associated with PD.

In another embodiment, a platform as provided herein delivers a zinc-dependent protease such as, for example, ADAM10 (ADAM metallopeptidase domain 10), ADAM17, or ADAM9 to cells of a subject diagnosed as having or suspected of having Alzheimer's disease, where delivery of zinc-dependent protease is beneficial to those specific cells. ADAM10, ADAM17, ADAM9, and other related proteases (e.g., other secretases of the ADAMs family of transmembrane proteins) are believed to cleave amyloid precursor protein (APP) and initiate proteolytic processing of APP. ADAM10 immunostaining has been shown to be reduced in the brains of AD patients (Bernstein et al., 2003, J Neurocytol 32(2):153-60). Neuronal overexpression of ADAM10 in transgenic mice carrying a human APP mutation (V717I), increased the secretion of sAPPα, reduced the production of Aβ and prevented its deposition in plaques (Postina et al., 2004, J Clin Invest 113(10):1456-64).

In another embodiment, a conjugate delivery platform as provided herein delivers Transcription Factor EB (TFEB). TFEB has been shown to reduce aggregation and neurotoxicity in a mouse model of Huntington disease. TFEB is also a therapeutic target for lysosomal storage disorders (LSDs) such as Pompe disease. For example, overexpression of TFEB reduced glycogen load and lysosomal size, improved autophagosome processing, and reduced accumulation of autophagic debris in vitro and in a mouse model of PD (Spampanato et al., EMBO Mol Med. 2013 May; 5(5):691-706).

In another embodiment, intracellular microRNAs (miRNAs) may be delivered to specific cells to serve as pain mediators. miRNAs are key regulators of gene expression. For instance, miRNA-let-7b induces rapid inward currents and action potentials in dorsal root ganglion (DRG) neurons.

Methods of Use. In one embodiment, the universal delivery platform of the present invention can be used to deliver a therapeutically effective amount of functional, therapeutic proteins to a subject in need thereof. By “subject,” we mean mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex. By “subject in need thereof,” we mean an animal or human subject who has been diagnosed with a disease or condition requiring treatment.

Specifically, the universal delivery platform provided herein can be used to deliver a therapeutically effective amount of heterologous, functional, therapeutic compounds to specific cells of a subject to treat specific conditions or diseases. For example, in one embodiment, the universal delivery platform of the present invention is useful for efficient delivery of a functional heterologous protein (e.g., β-lactamase) into the cytosol of a neuron to treat botulism.

As used herein, a “therapeutically effective amount” refers to an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors. For purposes of the present invention, “treating” or “treatment” describes the management and care of a patient for the purpose of combating the disease, condition, or disorder. The terms embrace both preventative, i.e., prophylactic, and palliative treatment.

“Treating” includes the administration of a compound of present invention to prevent the onset of the symptoms or complications, alleviating the symptoms or complications, or eliminating the disease, condition, or disorder. For instance, in one embodiment, the universal delivery platform of the present invention can be used to deliver therapeutic compounds that provide a “gain-of-function” benefit to LT-delivered therapies to repair molecular defects in host physiology. In some diseases, replacing function in even a small percentage of targeted molecules can be effective in slowing progression of a disease. For example, the majority of cystic fibrosis (CF) cases are from the ΔF508 mutation or premature termination codons (PTCs) that result in unstable mRNA and truncated CF transmembrane conductance regulator (CFTR). It has been shown that CF patients can see dramatic improvement in their condition by partially correcting the trafficking defect by facilitating exit from the endoplasmic reticulum of ΔF508-CFTR-mediated Cl(−) transport to more than 10% of that observed in non-CF human bronchial epithelial cultures, a level expected to result in a clinical benefit in CF patients. New strategies for correcting CF have identified protein targets, the overexpression or siRNA knockdown of which promotes ΔF508-CFTR processing/biogenesis and enhances ΔF508-CFTR channel activity (Collawn et al., Expert Rev Proteomics. 7(4): 495-506 (2010)).

The universal delivery platform of the present invention can be used to treat any disease or condition where a targeted delivery of therapeutic compounds can be useful, including, for example and without limitation, botulism, Huntington's disease, Parkinson's disease, Alzheimer's disease, Pompe disease, shingles, brain tumors (including gliomas), ALS, CF, spinal cord injuries, hemochromatasis, Crone's disease, cancer, HIV, leukemia, tuberculosis, and more. As shown in Table 2, the universal LT delivery system of the present invention can be adjusted to target specific conditions or diseases by adjusting which LT platform is used.

TABLE 2
LT platforms and targeted diseases

| | | | |
|---|---|---|---|
| LTIIa (camilid, E3 | GD1b/GT1b | Botulism | Light chain of BoNT |
| binding protein) | | | |
| LTIIa (BDNF) | GD1b/GT1b | Huntington's and | Increase expression of BDNF |
| | | Parkinson's disease | |
| LTIIa (Protease) | GD1b/GT1b | Alzheimer disease | Amyloid protein |
| LTIIa | GD1b/GT1b | Shingles | Herpes zoster |
| (kinase/protease) | | | |
| LTIIa (P53, PTEN, | GD1b/GT1b | Brain tumor, such | Tumor cell growth |
| pRb) | | as Glioma | |
| LTIIa (protease) | GD1b/GT1b | Spinal cord injury | PTEN |
| LTIIa (SOD1) | GD1b/GT1b | Amyotrophic lateral | Increase expression of functional SOD1 |
| | | sclerosis (ALS) | |
| LTI/CT (HFE) | GM1a | hemochromatosis | Increase functional expression of SOD1 |
| LTI/CT (NOD2, | GM1a | Crohn's disease | Increase expression of functional CARD15 |
| CARD15) | | | (also known as NOD2) |
| LTI/CT | GM1a | cancer | Increase expression of P53, PTEN |
| (P53, PTEN, pRB) | | | |
| LTI/CT (kinase, | GM1a | Virus, such as HIV, | Virus particle |
| protease) | | BK virus | |
| LT/CT/LTII | GD3 | acute leukemia | acute leukemia cells |
| (GMCSF) | | | |
| LTI/CT (kinase, | GM1a | TB | Intracellular bacterial within macrophage |
| protease) | | | |

The dose of the therapeutic compounds will depend on the condition being treated. The expectation is that conventional dosages, determined by the potency of the compound delivered, will be used in this delivery system. In addition, should the LT delivery system or its “cargo” prove immune stimulatory, the epitopes that stimulate the immune response will be mapped and eliminated as described by Pastan and coworkers who identified and eliminated the immune stimulatory epitopes within the binding domain of Pseudomonas aeruginosa exotoxin A (ETA) to facilitate the use of ETA as a therapeutic drug.

These and other features, objects and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### EXAMPLES

The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Example 1

### Materials and Methods

Plasmids construct. E. coli codon optimized sequence of LTIIa (accession number JQ031711) A subunit and B subunit were synthesized with dual IPTG-inducible T7 promoters (GenScript) and sub-cloned into the PET28a vector for expression. His₆ and HA₂ epitopes were added to the C terminus of the B subunit for purification and immunofluorescence detection, respectively. LTIIa A subunit and B subunit encode leader sequences for co-translational secretion into the periplasm. TEM1 β-lactamase (βlac, GenBank: AGW45163.1: amino acids 24-286) replaced the A1 subunit of LTIIa (amino acids 1-172), a 3X FLAG tag was down stream of the β-lac (βlac-LTIIa) producing βlac-LTIIa (FIG. 1). Site-directed-mutagenesis (S45A) produced βlacₙᵤₗₗ-LTIIa that lacked β-lac activity. DNA encoding βlac and βlacₙᵤₗₗ were also sub-cloned into DsRedmonoN1 to construct pβlac-Dsred and pβlacₙᵤₗₗ-Dsred, respectively. DNA encoding a single domain camelid antibody (VHH) specific for BoNT/A (ALc-B8, GenBank accession number FJ643070, amino acids 7-121) with a C-terminal 3X FLAG tag replaced the sequence encoding the A1 subunit of LTIIa (amino acids 1-172), yielding VHH-B8-LTIIa. Constructs were confirmed by DNA sequencing.

Protein expression and purification. Plasmids encoding LTIIa, f3 lac-LTlla, βlacₙᵤₗₗ-LTlla, βlac_{F}-LTlla and VHH-B8-LTIIa were transformed into E. coli BL-21(DE3). Transformants were grown overnight on LB agar plates containing 50 μg of kanamycin/ml, which were the inoculum for liquid cultures (LB, 400 ml) containing the same antibiotic. Cells were cultured at 37° C. to an OD600 of ˜0.6 when T7 promoter expression was induced with 1 mM IPTG. Cells were cultured overnight at 250 rpm at 16° C. Cells were pelleted and suspended in 20 mM Tris buffer pH 7.9 with 25% sucrose. Cells were treated with lysozyme (0.16 mg/ml in 0.1M EDTA) for 30 min. on ice, followed by addition of 70 mM MgCl₂ (final) and centrifugation at 5,000×g for 20 min to separate the soluble periplasm from the cells. His₆-tagged proteins were purified from the periplasm, using Ni2+-NTA resin (Qiagen). Purified proteins were dialyzed into 20 mM Tris buffer pH7.9 containing 20 mM sodium chloride and 40% glycerol. Aliquots were stored at −80° C.

Cells and reagents. Neuro-2a cells (ATCC; CCL-131) were cultured in Dulbecco's Modified Eagle Medium (DMEM, Invitrogen) with 10% fetal bovine serum (Invitrogen). Cells were transformed with pβlac-Dsred or pβlacₙᵤₗₗ-Dsred with lipofectamine LTX (Invitrogen) as suggested by the manufacturer. Rat primary cortical neurons were cultured as previously described. Briefly, rat E18 cortical neurons or rat E18 hippocampal neurons (BrainBits LLC) were cultured in Neurobasal medium (catalog no. 21103; Invitrogen) supplemented with 0.5 mM Glutamax-I (catalog no. 35050; Invitrogen), 2% B27 supplement (catalog no. 17504; Invitrogen), and Primocin (InvivoGen). Half of the medium was replenished every fifth day. Neurons were used at day 10 to 14 post-plating. Botulinum neurotoxin type D (BoNT/D) was isolated from C. botulinum strain 1873. The 150 kDa protein was purified using methods similar to those previously described for isolation of toxins from other BoNT serotypes. Specific activity in mice was 1.1×10⁸ LD₅₀ Units/mg.

βlac-LTIIa_{B} and LTIIa entry and translocation in Neuro-2a cells and primary cortical neurons. Neuro-2a cells were loaded with 10 μg/ml of the gangliosides GD1b or GM1a in DMEM with 0.5% FBS at 37° C. for 3 h. Cells were washed and incubated with 40 nM of β-lac-LTIIa_{B} or LTIIa in serum free DMEM at 37° C. for 60 min. Primary neurons were incubated with 40 nM of LTIIa, β-lac-LTIIa_{B} in neurobasal medium (supplemented with B27 and glutamax) at 37° C. or 4° C. for 60 min. Cells were washed with Hanks balanced salt solution (HBSS) and loaded with CCF2-AM dye (Invitrogen, 6X prepared as suggested by manufacturer to obtain a final concentration of 1X in HBSS). Samples were incubated at room temperature for 30 min and washed, and immunofluorescence staining was performed as described below.

Effects of VHH-B8-LTIIaB on BoNT-intoxicated neurons. Rat cortical neurons were incubated with 1 nM of BoNT/A or BoNT/D at 37° C. for 2 h, toxin was removed, and indicated amounts of VHH-B8-LTIIa_{B} (B8) were incubated with neurons at 37° C. for another 3 h. Cells were fixed and incubated with Alexa 647-wheat germ agglutinin for 30 min at room temperature (RT). Immunofluorescence (IF) staining, as described below, detected BoNT/A cleaved SNAP25 (SNAP25c) or intact VAMP2 in BoNT/D treated cells. Cleavage of SNARE substrates was quantified using the ratio of fluorescence intensities SNAP25c/WGA for BoNT/A intoxicated cells and VAMP2/WGA for BoNT/D intoxicated cells. In other parameters of this experiment, neurons were incubated with 1 nM of BoNT/A or BoNT/D with the indicated amounts of VHH-B8-LTIIa at 37° C. overnight and SNARE substrates cleavage was evaluated as described above.

IF staining. Cells were fixed with 4% (wt/vol) paraformaldehyde in DPBS for 15 min at RT, washed twice with DPBS, permeabilized with 0.1% Triton X-100 in 4% formaldehyde in DPBS for 15 min at RT, incubated with 150 mM glycine in DPBS for 10 min at RT, washed with DPBS twice, and subjected to IF staining. Treated cells were incubated in blocking solution (10% normal goat serum, 2.5% cold fish skin gelatin [Sigma], 0.1% Triton X-100, 0.05% Tween 20 in DPBS) for 1 h (RT), followed with primary antibody (anti-HA antibody (Roche) and anti-FLAG antibody (Sigma)) in antibody incubation solution (5% normal goat serum, 1% cold fish skin gelatin, 0.1% Triton X-100, 0.05% Tween 20 in DPBS) overnight at 4° C. Cells were washed three times with DPBS+0.05% Tween20, and incubated with goat or rat IgG Alexa-labeled secondary antibodies (Molecular Probes) in antibody incubation solution for 1 h (RT). Cells were washed 3 times, fixed with 4% paraformaldehyde in DPBS for 15 min (RT), and washed with DPBS. Mounting reagent Citifluor AF-3 (Electron Microscopy Sciences) was added, and images were captured with a Nikon TE2000 total internal reflection fluorescence (TIRF) microscope equipped with a CFI Plan Apo VC 100×oil, numerical-aperture (NA) 1.49 type objective using a Photometrics CoolSnap HQ2 camera. Image analyses were performed using Nikon NIS-Elements. Figures were compiled using Canvas X (ACD Systems).

Data analysis and statistics. Images were generated with equal exposure times and conditions. Image intensity analysis and colocalization analysis were performed using Image J 1.48b (NIH). Data statistical analysis was performed using GraphPad Prism 5.0 (GraphPadSoftware, CA.).

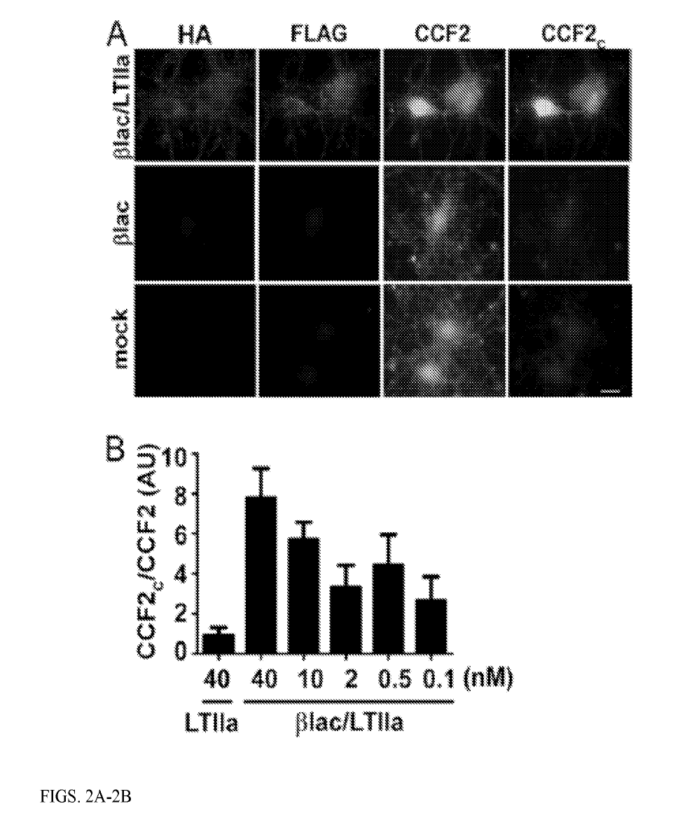

Results. βlac is catalytically active in Neuro2a cells. A FRET-based assay was used to measure βlac in neuronal cells. Briefly, cells were incubated with CCF2-AM, a heterocyclic small molecule, which passes across cell membranes and is converted by cytosolic host esterase to CCF2. Intracellular CCF2 has FRET properties. Upon excitation at 409 nm CCF2 shows a FRET emission at 520 nm (green), but β-lac cleaves CCF2 and the FRET signal is lost and the emission shifts to 447 nm (blue). Neuro-2a cells were transfected with p β-lac-Dsred or pβlacₙᵤₗₗ-Dsred and assayed for excitation/emission profile of CCF2. Cells expressing βlac-Dsred, but not βlacₙᵤₗₗ-Dsred, showed blue fluorescence, which supported a βlac-dependent cleavage of CCF2 (FIG. 2).

βlac-LTlla is assembled into an AB protein. SDS-PAGE analysis assessed the assembly βlac-LTlla into an AB protein complex. Affinity purified LTIIa, βlac-LTIIa and βlacnull-LTIIa comprised two bands corresponding to the size of the A or the βlac-A2 within the LTIIa chimera and B subunit at A:B ratios of 5.4 and 5.2. Since the proteins were purified with an affinity epitope located on the B subunit, the detection of the A subunits of LTIIa and βlac-LTIIa_{B} supports A-B assembly within βlac-LTIIa β (FIG. 1).

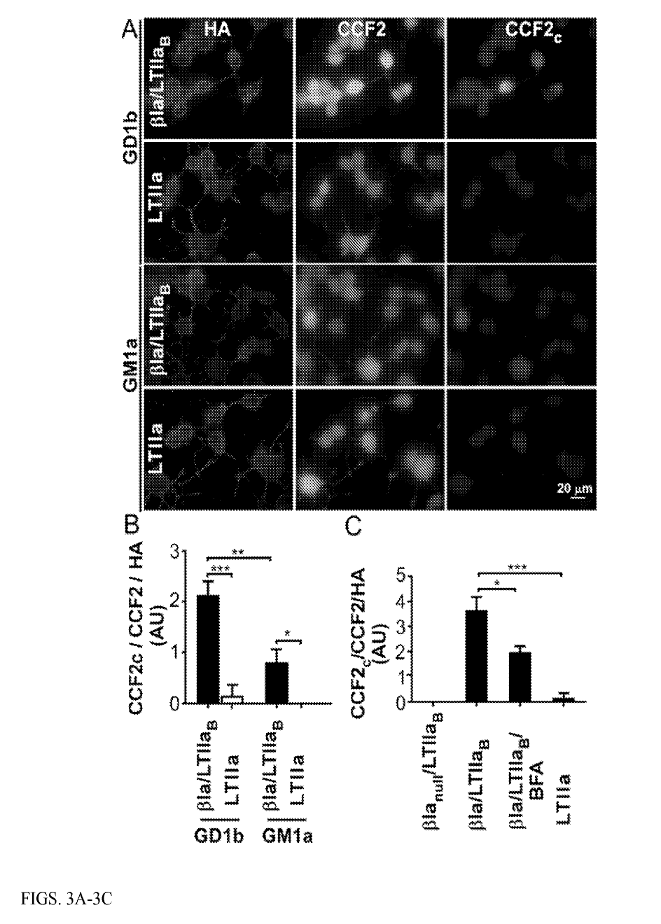

LTIIa delivers functional cargo (β-lac) into neurons. To test if LTIIa can deliver a functional, heterologous protein into neurons, the chimeric LTIIa derivative, β-lac-LTIIa_{B} was engineered. Proper delivery of enzymatically active βlac into primary rat cortical neurons was examined by the CCF2 FRET cleavage assay. Incubation of β-lac-LTIIa_{B} with primary rat neurons showed CCF2 cleavage (cyan), while incubation with LTIIa did not yield CCF2 cleavage (FIGS. 3A-C). This result supports the ability of LTIIa to deliver a functional, heterologous protein (β-lac) into neurons. Titration of β-lac-LTIIa_{B} yielded a proportional amount of CCF2 cleavage, with detection of β-lac activity in cells treated with amounts as low as 0.1 nM of β-lac-LTIIa_{B}.

To address if heterologous protein delivery had “off-target” effects, LTIIa was evaluated for the preferred delivery of β-lac by complex gangliosides and if delivery was via a BFA-sensitive pathway. LTIIa binds the ganglioside GD1b with the highest affinity and binds GD1a, GT1b, GQ1b, GM1, and GD2 with lower affinity. GM1a in T84 cells does not mediate signal transduction by LTIIa. Neuro-2a cells do not express complex gangliosides such as GD1b, however, upon incubation with exogenous gangliosides, their membranes can be loaded with gangliosides. Upon incubating membranes with exogenous gangliosides, LTIIa delivered β-lac over 2-fold more efficiently into GD1b treated cells than GM1a treated cells, consistent with the higher affinity of LTIIa for GD1b relative to GM1a. BFA is a fungal metabolite that inhibits vesicular transport in the secretory pathway and vesicular exchange between endosomes and Golgi cisternae/ER in eukaryotic cells. BFA inhibited βlac translocation by LTlla in both GD1b loaded Neuro-2a cells and rat primary neurons, as reported for native LT. These experiments indicate that β-lac-LTIIa_{B} does not have “off target” trafficking properties.

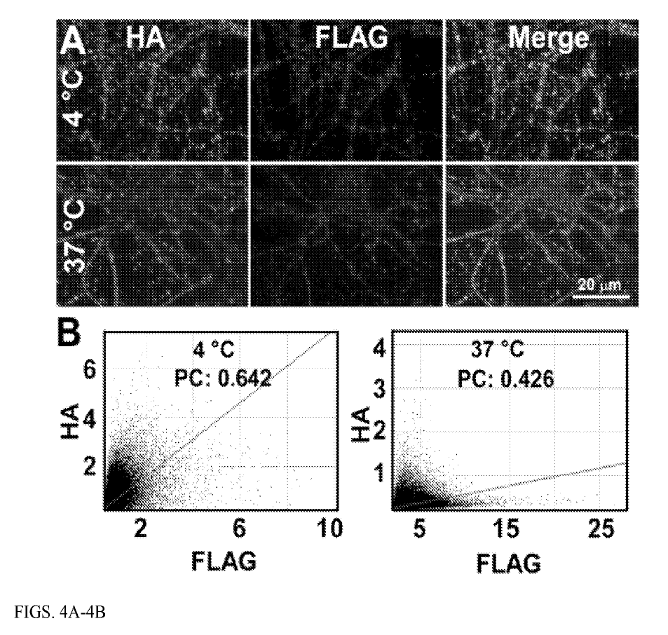

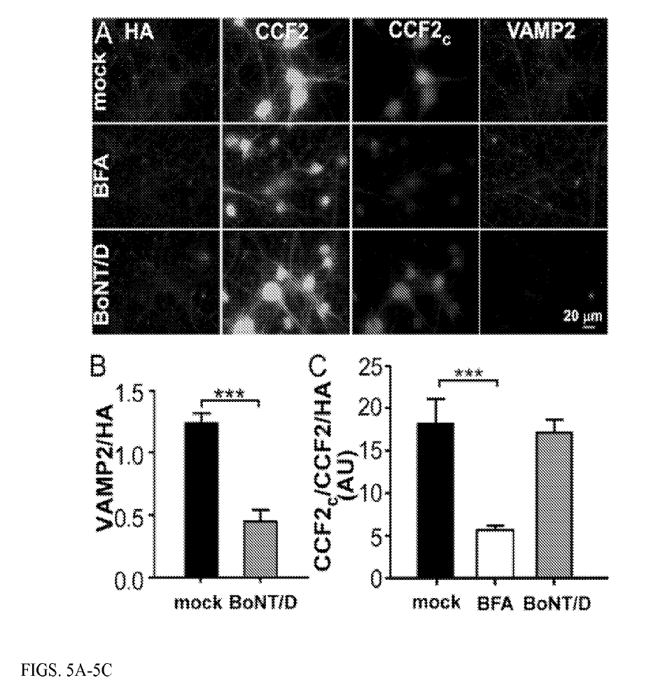

To further investigate the trafficking of the β-lac cargo and B subunit of β-lac-LTIIa_{B}, a FLAG epitope was engineered on the C terminus of β-lac to detect cargo localization, while an HA epitope allowed B subunit detection. The FLAG tag did not affect the translocation of cargo by LTIIA, as determined by the FRET based cleavage assay for beta-lac activity (FIGS. 4A-B). FLAG and HA epitopes had a Pearson's Coefficient (PC) of 0.64 upon incubation of β-lac-LTIIa_{B} on neurons at 4° C., indicating the baseline of co-localization when cell-bound. Upon incubation at 37° C. for 1 h, the PC of FLAG/HA decreased to 0.43, showing a segregation pattern between the two epitopes. This supports the neuronal cell entry and separation of cargo from the B subunit of LTIIa within neurons (FIGS. 4A, 4B).

LTIIa delivers functional cargo (β-lac) into BoNT/D-intoxicated neurons. Botulinum neurotoxin cleaves SNARE proteins to prevent synaptic vesicle fusion. To determine if LTIIa can serve as a therapy delivery platform for BoNT intoxicated neurons, the ability of LTIIa to deliver β-lac into BoNT/D intoxicated neurons was measured by the CCF2 FRET assay. Rat primary neurons were incubated overnight with 2 nM BoNT/D to cleave endogenous VAMP2, which was confirmed by IF staining (FIGS. 5A-5C). Incubation of these neurons with β-lac-LTIIa_{B} resulted in CCF2 cleavage similar to control neurons that were not exposed to BoNT/D. This indicates that entry and translocation of βlac by LTIIa is not inhibited in BoNT/D intoxicated neurons (FIGS. 5A, 5C). Thus, LTIIa may be a useful delivery platform for BoNT therapeutics.

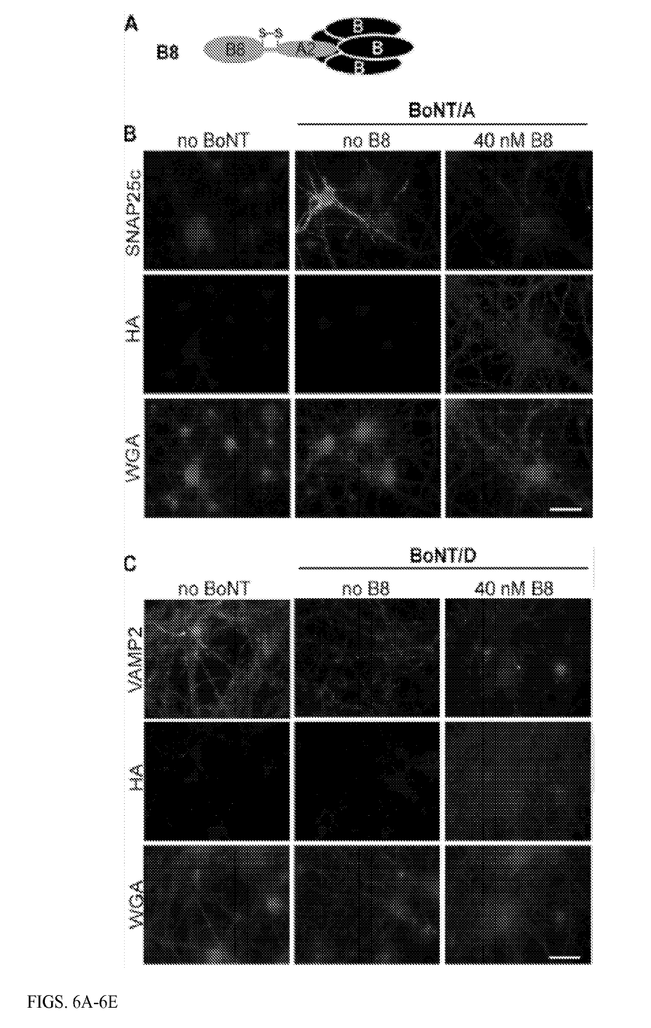

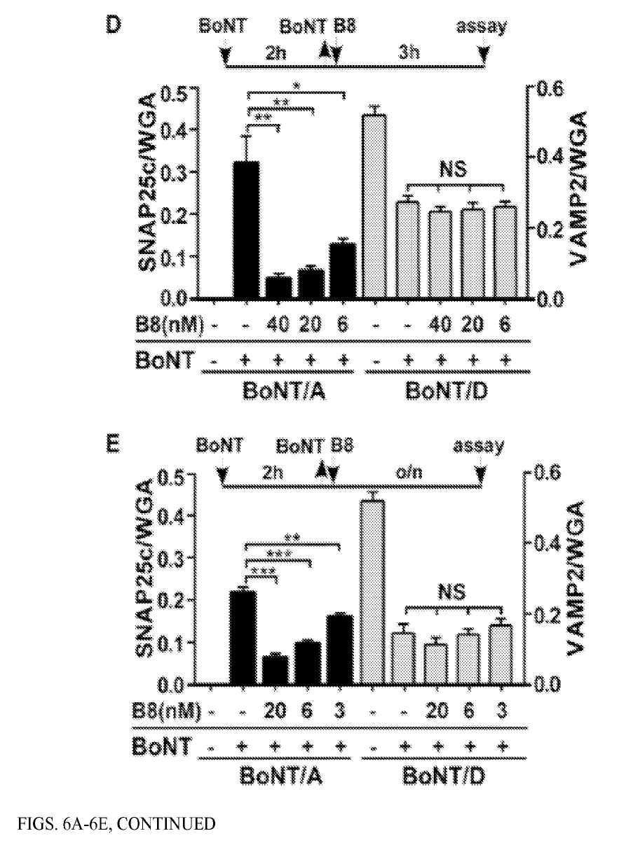

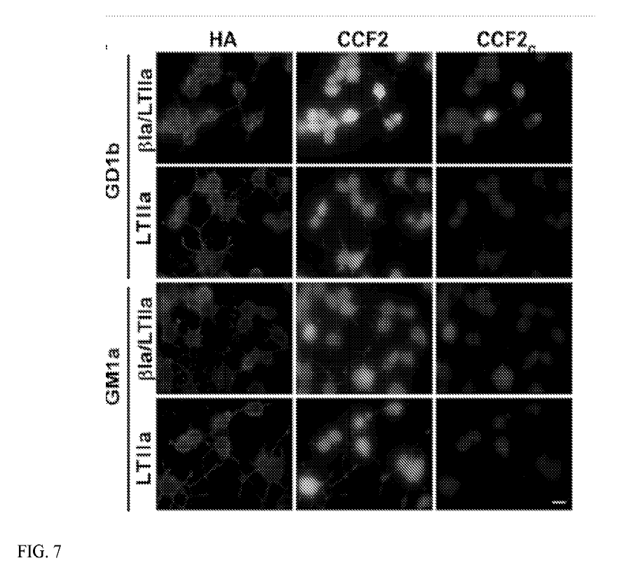

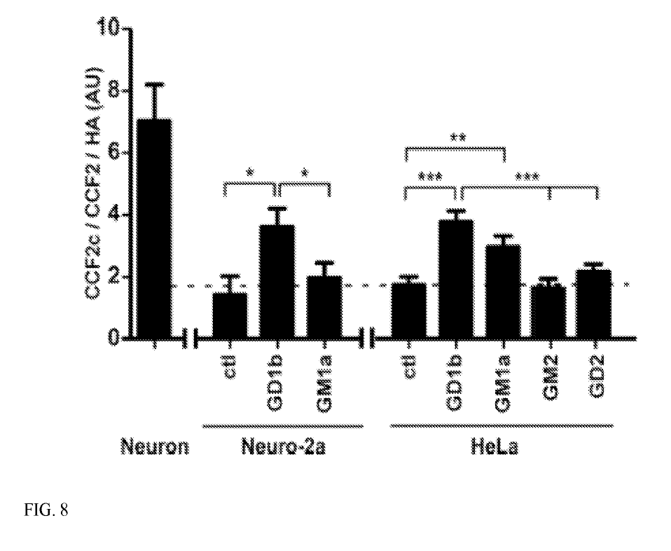

VHH-B8-LTIIa inhibited BoNT/A cleavage of SNAP25 in rat cortical neurons. In order to assess the utility of LTIIa as a delivery platform for anti-botulism therapeutics, an LTIIa chimera was engineered where the A1 subunit was exchanged with a single chain variable region camelid antibody that has previously been shown to target and inhibit the LC of BoNT/A (B8) (termed VHH-LTIIa) (FIG. 6A).

Primary cortical neurons were incubated with 1 nM BoNT/A or BoNT/D for 2 h at 37° C., washed, and incubated with varying amounts of VHH-LTIIa for 3 h and then assayed for cleaved SNAP25 (BoNT/A treated cells) or full length VAMP2 (BoNT/D treated cells) (FIGS. 6B, 6C). Cleaved SNAP25 was detected in cells treated with BoNT/A alone, while BoNT/A intoxicated cells also treated with VHH-LTIIa showed a dose-dependent reduction of cleaved SNAP25 (FIGS. 6D, 6E). Controls showed that VHH-LTIIa inhibition was serotype dependent, since VHH-LTIIa did not inhibit the cleavage of VAMP2 by BoNT/D. VHH-LTIIa also inhibited BoNT/A protease activity in cells incubated overnight, showing the potential longevity of the effect of this BoNT/A therapeutic. This is the first example of the delivery of a functional therapy into a BoNT-intoxicated neuron.

Discussion. In this example, LTIIa was chosen for development of a neuron specific therapeutic delivery system, based on the high affinity of LTIIa to the complex ganglioside GD1b, which is enriched in neuronal tissues. LTIIa delivered β-lac as a reporter cargo into both Neuro-2a cells loaded with exogenous GD1b (FIG. 5) and primary cortical neurons. Translocation of β-lac was detected in all of the rat primary cortical neurons (FIG. 5) and Neuro-2a cells (FIG. 6). LTIIa has low affinity to GD1a, GT1b, GQ1b, GM1, and GD2 and high affinity to GD1b in vitro. Thus, LTIIa can be developed as a delivery platform for neuronal cells. Future studies will optimize neuronal specificity by engineering LTIIa to bind gangliosides that are unique to neurons.

β-lac-LTIIa_{B} was examined as a neuron specific delivery platform. The βlac cargo entered and translocated in neurons intoxicated by BoNT/D, supporting the utility of LTIIa as a therapeutic delivery platform post BoNT intoxication (FIG. 8). When a therapeutic camelid single domain protein VHH-B8 was delivered to BoNT/A intoxicated neurons via LTIIa platform, VHH-B8 inhibited SNAP25 cleavage by BoNT/A (FIG. 8).

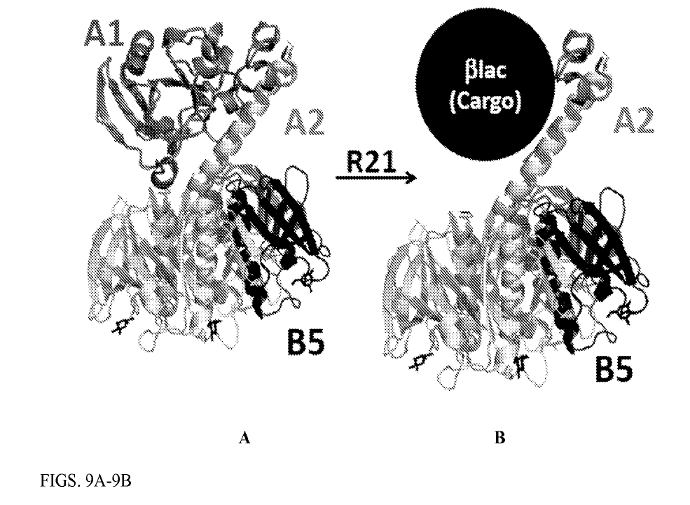

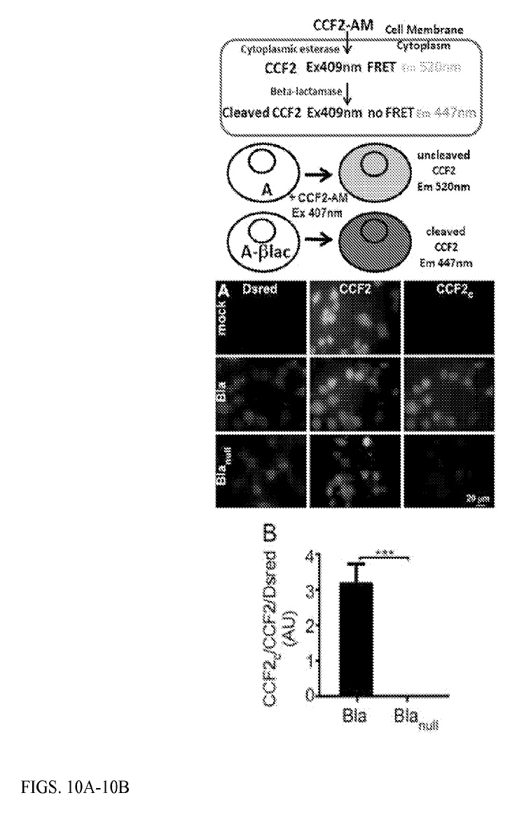

Light chains of BoNTs remain active inside neurons, which can cause flaccid paralysis for up to six months. The delivery of therapeutic cargo is essential to neutralizing BoNT LCs inside neurons. VHH-B8-LTIIa not only inhibited SNAP25 cleavage when incubated for 3 h with neurons intoxicated by BoNT/A, but also when it was incubated overnight with neurons along with BoNT/A (FIG. 9), suggesting an approach as a therapeutic after BoNTs intoxication. The fact that VHH-B8-LTIIa did not affect VAMP2 cleavage by BoNT/D, further confirms the specificity of this therapeutic platform (FIG. 8).

Progress has been made to develop BoNT vaccines and neutralizing antibodies in the past two decades, however, they cannot provide therapy after BoNT has entered neuronal cells. Our study is the first time LTIIa has been employed as a platform to deliver with specificity a therapeutic cargo to neurons to neutralize intracellular BoNT light chain activity.

The LTIIa-derivatives of the present invention provide a new approach to deliver therapeutics to treat neurodegenerative diseases, BoNT intoxication, and latent virus infection in neurons.

### Example 2

### Characterizing the Potency and Neuronal Specificity of the LTII Delivery Platform

The LTIIa delivery platform described in Example 1 assembled heterologous proteins into an AB5 conformation and delivered two independent cargos, β-lac and camelid, into the cytosol of a neuron via a BFA sensitive pathway. This showed that the LTIIa-derivative trafficked like native LTIIa and not through an “off target” mechanism. Example 1 also shows that LTIIa cargo delivery was more efficient with GD1b as the host receptor relative to GM1a, showing the neuron specificity of LTIIa delivery.

In this example, the inventors characterize the potency and neuronal specificity of the LTII delivery platform. First, the inventors inactivate the immune response of the B subunit of LT. Site-Directed Mutagenesis (QuickChange) will engineer changes into DNA encoding the B subunit of LTI to make a H57S substitution to eliminate an immune modulating activity. The B subunits of LTIIa and LTIIb will be engineered to eliminate a TLR1/2 response region (residues 68-74 of LT-BIIa) making the L73A/S74D substitutions that are in direct contact with TLR2; where each individual mutations reduces LTB-mediated cytokine activation in macrophages to near background levels. Mutated B subunits and the holo-toxin LTII forms will be assessed for cytokine stimulation, using THP-1 derived monocytes. Solid phase binding will test if mutations that eliminate TLR2 binding, while retaining ganglioside binding. Together, these experiments will eliminate an intrinsic immune modulating property of the B-subunits of the LT toxins.

Determine the tropism of cargo delivery by LTIIa (binds GD1b), LTIIb (binds GDIa), and LTI (binds GM1a) for cellular subtypes of neurons. We have shown that LTIIa delivered cargo more efficiently into primary cortical neurons than cultured neuronal cells or non-neuronal cells such as HeLa cells, supporting the tropism of LTIIa for primary neurons. This example will address the tropism of the LTII toxins for subsets of neurons, including primary neuronal cells (spinal cord, motor neurons, hippocampal, and cortical neurons, purchased from BrainBits), cultured neuronal cells (PC-12, ATCC CRL-1721, Neuro-2a, ATCC CCL-131, and C6-Glial cells, ATCC CCL-107), and non-neuronal cells (epithelial cells, HeLa, ATCC CCL-2 and endothelial cells, ATTC PCS-100-010). Cells will be cultured as monolayers and incubated with; βlac-LTIIa, βlac-LTIIb or βlac-LTI. The translocation potency of each LT-derivative will be determined, using the β-lac as a reporter as described in the preliminary results. Cultured neuronal and non-neuronal cells will be loaded with individual complex gangliosides and tested for sensitivity to βlac-LTIIa, βlac-LTIIb or βlac-LTI. These experiments will establish a potency array for the LTII-derivatives for subsets of neurons and the ganglioside preference for cargo translocation. This will be an initial indication of the potential heterogeneity of sensitivity to the LT-derivatives within subset of neurons that could facilitate the identification of preferred targets of individual LT-derivatives within unique regions of the brain.

Measure efficiency of cell binding and intracellular trafficking. LTIIa (high affinity for GD1b), LTIIb (high affinity for GDIa), and LTI (high affinity for GM1a) will be engineered with and without catalytic activity and with unique epitopes on the A subunit (FLAG) or B subunit (HA) to track the intracellular movement of the two domains. Pearson Coefficients (PC) which measures the co-localization between the two probed epitopes will establish association of the A and B subunits of the LTII-derivatives on the cell membrane (bound form) and then assess association of the A and B subunits as the LTII-derivatives progress into the cell to establish the efficiency of intracellular delivery of the cargo protein.

Measure efficiency of cargo translocation into the cell cytosol. βlac-LTIIa, βlac-LTIIb or βlac-LTI will be used to measure the efficiency of cargo delivery into the cell cytosol as the cleavage of CCF2 (a quantitative assay of βlac translocation to the cytosol). The sensitivity of cargo translocation to Brefeldin A (BFA) treatment will determine if trafficking and translocation proceeds through the Golgi, as observed for native LT. Thus, BFA sensitivity is an assessment of potential “off target” trafficking by a LTII-derivative. The cultured neuronal cells, such as Neuro-2a, possess limiting complex gangliosides and the plasma membrane of these cells will be “loaded” by incubation with exogenous gangliosides, including GT1b, GD1b, GD1a, and GM1a to assess how trafficking via each class of ganglioside influences trafficking and translocation. Loading efficiency of gangliosides will be established using ganglioside specific antibodies (α-GT1b antibody, Millipore) and toxins that bind to specific classes of gangliosides (CTB which binds exclusively to GM1a).

Determine how intracellular trafficking affects the potency of cargo delivery by LTII. The C-terminal RDEL sequence of A2 targets LTII to the endoplasmic reticulum to facilitate the translocation of the A1 subunit into the cytoplasm and deletion of the RDEL sequence redistributes the trafficking of LT into other delivery pathways. RDEL will be deleted from A2 the LTII(a-c) to determine the effects on LTII entry and β-lac translocation efficiency. Pearson Coefficients (PC) will measure the co-localization between to probed epitopes and establish the entry progress of the LTII-derivatives and efficiency of the intracellular delivery β-lac cargo into cytosol. The sensitivity of cargo translocation by Brefeldin A will determine if the trafficking processes through the Golgi and if alternative entry pathways provide an efficient translocation or change in the site of delivery. This is of interest, since in earlier studies βlac-Cholera toxin-derivative delivered cargo to a different subcellular location (central) than the βlac-LTIIa-derivative (peripheral, extending into the dendrites and axon) within primary cultured neurons. Assessment of BFA sensitivity will also determine potential “off target” trafficking by the various LTII-derivatives. Together, these experiments will measure the influence of the C-terminal RDEL sequence on the intracellular trafficking and efficiency of cargo translocation.

Anticipated Results. We anticipate that mutations in the TLR1/2 binding region of the LTBII toxins and in the immune modulating region with LTI will reduce the association with TLR1/2 receptor and reduce cytokine production to control (unstimulated) levels. We do not anticipate that these mutations will affect ganglioside binding, the pentameric state of the B subunits, or the ability to assemble with the A2 linker.

LTII-derivatives will possess unique potency and trafficking patterns upon entry into each unique subset of primary neuron and a unique association with the cultured neuronal cells with each class of ganglioside. Cargo translocation, delivery of βlac into the cytosol, potential of LTIIa, LTIIb, and LTI and how gangliosides influence cargo translocation efficiency will be unique. Note, by establishing the preferred binding of LTIIa, LTIIb, and LTI for GT1b, GD1b, GD1a, and GM1a in cultured Neuro-2a cells, one can correlate trafficking properties and efficiency of cargo translocation by individual classes of gangliosides by each LT-derivative. This will define how association with specific gangliosides influences trafficking and cargo transport. We cannot predict if LTIIa or LTIIb will be the most efficient at delivering cargo into neurons, but we do predict that either of the LTII-derivatives will be more efficient at delivering cargo relative to LTI, based on earlier studies with LTIIa and CT.

Deletion of the C-terminal RDEL from the C terminus of A2 is anticipated to distribute LTIIa into an endosome entry pathways that may enhance efficiency and location of cargo translocation. We initially predict that removal of the RDEL will delay delivery of cargo into the cytosol, but not the efficiency of translocation and will change the site of intracellular localization of cargo, which may influence the potency of a therapy. This prediction is based upon earlier studies with cholera toxin, which observed differential intracellular trafficking, and A subunit translocation with the removal of the RDEL sequence.

Alternative Approaches. We do not anticipate problems engineering point mutations within the B subunits, but if this is not efficient, overlap PCR mutagenesis can be used to make these mutations. Should the point mutated B subunits retain residual immune activity, a third point mutation will be engineered within the TLR1/2 binding region. The triple mutated B subunit will be tested for biological properties to determine other functions (ganglioside/pentamerization/A2 linker assembly) were also affected. There is some concern that the mutations that reduce TLR2 modulation also reduce the ability to assemble the A2 linker into the B oligomer. Should A2 assembly be affected more conservative mutations will be engineered at residues 73/74 of the B subunit to facilitate A2 linker, but eliminate immune stimulation through TLR1/2. For example, since the TLR-2-B subunit interactions are primarily hydrophobic, class changes in hydrophobicity will be used to eliminate TLR1/2 interactions without affecting A2 linker-B subunit interactions.

Should the class of ganglioside not influence the trafficking or cargo translocation efficiency of the LTII-derivatives, the LT-II derivative that assembles heterologous cargo most efficiently will be utilized in subsequent experiments. Further, should deletion of the RDEL disable the A subunit-B oligomer interaction, conservative substitution will be engineered to exchange the RDEL that eliminates targeting to the Golgi, but retains subunit association.

This example will optimize the LTII-derivative as a protein delivery platform, eliminating potential cytokine stimulation, and define how specific ganglioside receptor interactions and intracellular trafficking influence the efficiency and site of cargo translocation. Having this catalogued set of LTII-derivatives will provide the opportunity to test the translocation efficiency of the LT-II derivatives on specific cargos analyzed in other sections of this application.

### Example 3

### Optimizing the Neuronal Binding Specificity of LTII for Neurons

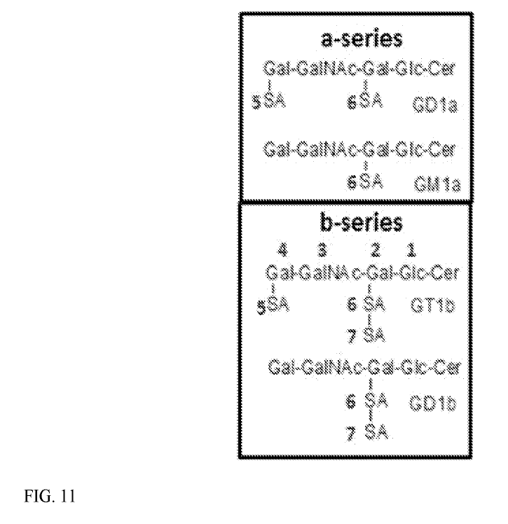

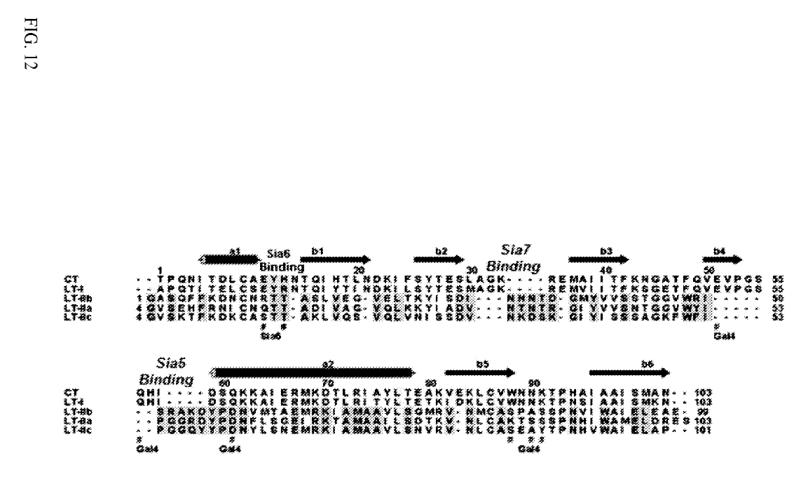

In this example, we show how the specificity and potency of the B subunit binding to neurons can be enhanced by binding complex gangliosides. There are several complex gangliosides enriched in the brain (FIG. 11), including GT1b, GD1b, and GD1a, while GM1a is present in membranes of neurons and non-neuronal sources. The goal of this aim is to reduce “off-target” effects of LTII therapies by targeting the complex ganglioside GT1b. LTIIa and LTIIb will be engineered to bind GT1b.

Specifically, for LTIIa, change the GD1b binding site to a GT1b binding site by adding the sia5 sialic acid binding pocket from LTIIb. Further, for LTIIb, change the GD1a binding site to a GT1b binding site by adding the sia7 sialic acid binding pocket from LTIIa.

Methods. Enhance binding of LTIIa and LTIIb to the complex ganglioside GT1b. Mutagenesis will generate a binding pocket within LTIIa for 5′ sialic acid and LTIIb for 7′ sialic acid to increase affinity for GT1b. Since LTIIa naturally binds GD1b, adding the capacity to bind GT1b would require the addition of a Sia5 binding site onto the GD1b binding site. The residues that comprise a Sia5 site were derived by modeling the Cholera toxin-GMa structure (PDB: onto LTIIb (PDB: 1TII). This analysis predicted that residues 50-59 comprised a Sia5 binding site pocket while residues 30-35 comprised a Sia7 binding site (FIG. 12). Thus, mutations will be engineered into DNA encoding the B subunit of LTIIa will be mutagenized to introduce a Sia5 site, 51- [symbol] -56 (SEQ ID NO:1)→ [symbol] (SEQ ID NO:2) and DNA encoding the B subunit of LTIIb will be mutagenized to introduce a Sia7 site, 30- [symbol] -35 (SEQ ID NO:3) [symbol] (SEQ ID NO:4) (Table 3).

TABLE 3
Modifications to LT.

| | Modification to LTIIa | Modification to LTIIb |
|---|---|---|
| | to add the | to add the |
| | sia5 binding | sia7 binding |
| | site from LTIIb à a | site from LTIIa à a |
| | GT1b binding site | GT1b binding site |
| LT | Sequence of Sia5′ | Sequence of Sia 7′ |
| | site | site |
| IIa | 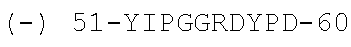 | 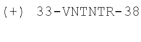 |
| | (SEQ ID NO: 5) | (SEQ ID NO: 4) |
| IIb | 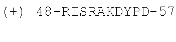 | 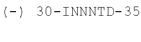 |
| | (SEQ ID NO: 6) | (SEQ ID NO: 3) |

The ganglioside binding affinities for LTII-derivatives generated as described will be measured by solid phase assay to establish changes in affinity and for purified gangliosides. In addition, changes in ganglioside specificity will test for correlations in changes in the efficiency of entry and translocation for βlac from the mutagenized βlac-LTII-derivatives in ganglioside-enriched Neuro2a cells and rat primary cortical neurons as described above.

Anticipated Results. Acquisition of GT1b binding should increase the potency of the LTIIa and LTIIb delivery platforms, since GT1b will have several additional direct ganglioside interactions relative to the native LTIIs. We anticipate that mutagenized LTIIa will bind GT1b with a higher affinity than modified LTIIb, since LTIIa has an intrinsic high affinity for ganglioside than LTIIb. Thus, this analysis will provide an opportunity to measure how ganglioside affinity affects delivery potency. We anticipate that the intrinsic binding of LTIIa and LTIIb for GM1a will not have to be modified since others have shown that LTIIa binding to GM1a does not lead to a productive intoxication.

Alternative Approaches. The primary concern is that additional modifications could be needed to provide a pocket for LTIIa to bind Sia5 of GT1b. If the two sets of mutations are not sufficient to allow GT1b binding, the pocket comprising the Sia5 site will be increased in size by mutating LTIIa Y49R and I39M, which lie adjacent to the Sia5 pocket. Another concern is that the two Ala mutations proposed are not sufficient to disrupt LTIIa-Sia6 interactions to reduce affinity for GM1a. In this case, bulkier hydrophobic residues will be introduced that are made to disrupt the hydrogen bond between T17 to reduce Sia6-LTIIa interactions. If the intrinsic binding of the modified LTIIa or LTIIb for GM1a, we have mapped the region of the B subunits for Sia6 interactions to the CT equivalents of E11 and H13 (FIG. 13) and will mutagenize this region to reduce the affinity of modified LTIIa and LTIIb for GM1a.

By optimizing the binding properties of the LTIIa and LTIIb to engineer the B subunits to bind specific classes of gangliosides, we will determine how trafficking to each ganglioside affects the efficiency of delivery and location of the therapy.

### Example 4

### Engineer LTII to Delivery BoNT Therapies to Engineer a Pan-Serotype BoNT Therapy

This example will extend our determination that LTIIa delivered a single chain camelid antibody (B8) as a therapy against BoNT intoxication. The therapies neutralize BoNT in vitro and/or when expressed within cells, but not used as a therapy. The therapeutic approaches to neutralize intracellular LC activity include delivery of: a) single chain camelid (VHH) β-LC antibodies (˜14 kDa). Shoemaker and colleagues showed that the VHHs bound BoNT-LC at high affinity (K_{d}˜1 nM), inhibited BoNT protease activity (K(i)˜1 nM), and retained binding specificity and inhibitory functions when expressed within mammalian neuronal cells.

We and other have mapped the interactions between BoNT-LC and substrate, identifying specific residues that are involved in binding and hydrolysis and specific mutations within the substrates that reduce and increase affinity of the LC for the substrate, using this information non-hydrolysable high affinity SNAP25 and VAMP2 substrates will be engineered as intracellular LC inhibitors

Fusions of the high affinity SNARE substrate inhibitors and camelid single-domain antibody therapies to address the engineering of a pan-serotype BoNT therapy, and d) addition of the E3 ligase-binding domain of βTrCP to target therapy-LC complexes to the ubiquitin degradation pathway. Shoemaker and colleagues have observed that the addition of the E3 ligase-binding domain F Box(175-293) enhances the clearance of intracellular VHH. When logistical, initial experiments will establish the therapeutic potency against BoNT intoxication in in vitro SNARE cleavage and cultured primary neurons. The most potent therapies will be tested in a mouse model of BoNT intoxication.

The general strategy for the engineering of the cargo-LT derivatives is the replace amino acids 1-170 of the A subunit of the targeted LT (LTIIa, LTIIb, or LTI) as was conducted by the engineering the βlac or β-BoNT/A B8 camelid single chain antibody-LTIIa chimeras (FIGS. 16A-16C).

Single chain camelid β-LC antibodies (VHH) to neutralize BoNT-A and BoNT-B. Table 4 shows the primary amino acid sequences for the β-LC-camelid VHHs that will be used as therapeutic cargo delivered by LTIIa. BoNT serotype specificity will be tested with a heterologous serotype BoNT.

TABLE 4
Camelid single chain antibody amino acid sequence.

| | | |
|---|---|---|
| | | Affinity for |
| Camelid | Camelid single chain antibody amino acid sequence | LC |

LC-A

| | | |
|---|---|---|
| B8 | SGGGLVQPGGSLRLSCAASGSIFSIYAMGWYRQAPGKQRELVAAISSYGSTNYADSVKGRFTI | High for |
| (115 aa) | SRDNAKNTVYLQMNSLKPEDTAVYYCNADIATMTAVGGFDYWGQGTQVTVSS | LCA |
| | (SEQ ID NO: 7) | |
| H7 | SGGGSVQPGGSLRLSCAAIGSVFTMYTTAWYRQTPGNLRELVASITDEHR-TNYAASAEGRFT | Mid for |
| (112 aa) | ISRDNAKHTVDLQMTNLKPEDTAVYYC---------KLEHDLGYYDYWGQGTQVTVSS | LCA |
| | (SEQ ID NO: 8) | |

LC-B

| | | |
|---|---|---|
| B10 | SGGGMVQPGGSLRLSCAASGFTFSTYDMSWVRQAPGKGPEWVSIINAGGGSTYYAASVKGRF | High for |
| (121 aa) | AISRDNAKNTLYLQMNNLKPEDTALYYCARVASYYCRGYVCSPPEFDYWGQGTQVTVSS | LCB |
| | (SEQ ID NO: 9) | |

SNARE Binding Inhibitors (high affinity(HAf-SNAP25 and HAf-VAMP2) as BoNT therapies. We have previously determined optimal SNARE binding domains for each BoNT serotypes. SNAP25 (residues 141-206) will encode three mutations that do not influence SNARE binding, but block BoNT serotypes A, E, and C cleavage (R198A, 1181E, and A199D). A glycine will also be inserted between the P1-P1′ residue for BoNT/A cleavage since this addition enhanced affinity for LC/A by ˜10-fold. VAMP2 (residues 10-94) will encode four mutations that do not influence LC-SNARE binding, but block BoNT serotypes B, D, F, and G cleavage (F77A, L60A, K59A, and G82D) and four mutations that enhance affinity for LC/B ˜70-fold (V42A,V43A,D44A,I45A), respectively. Note, this therapy can be modified with the identification of “new” BoNT serotypes such as the “new” BoNT/F and presumed BoNT/H.⁸ Utilization of high affinity non-hydrolysable SNARE substrates for multiple LC serotypes should make these substrates preferred relative to the native SNARE proteins.

Pan-BoNT therapy: (HAf-SNAP25-HAf-VAMP2-LTII) and BoNT/A-BoNT/B therapy. (α-LC/A-α-LC/B) Overlap PCR will engineer the SNAP25-VAMP2-LTII and αLC/A-LC/B-LTII gene fusions that will be assembled into LTII towards the generation of the a pan-serotype BoNT therapy. Fusion of the SNARE proteins or camelid α-LCs VHHs may include (GGGGS)₃ peptide linker to facilitate flexibility.

Enhance therapeutic potency with additions of cis-E3 ubiquitin ligase F box. The gene encoding the F box (an E3 ubiquitin ligase binding domain, residues 175-293, of βTrCP will be fused to SNAP25-VAMP2 and the β-LC inhibitors to include an active basis (ubiquitin targeting) to clear intracellular BoNT LCs.

DNA encoding the therapeutic agents will be engineered into the LTII platform, by overall PCR. DNA will be sequenced for validation. Translocation efficiency and neutralizing potency of the therapeutic-LTIIa derivatives will be assessed by targeting primary neurons that have been previously been intoxicated with BoNT/A or BoNT/B and measuring the Pearson's Coefficients or measuring cleavage of intracellular SNARE proteins, respectively. Controls for non-specific effects will include the titration of derivatives of the SNAP25-LTII and VAMP2-LTII against BoNT/A or BoNT/B intoxication, respectively, where the anticipated outcome would be protection to BoNT challenge by homologous BoNT serotype therapy, but not by the heterologous serotype therapy. The ganglioside specificity of the LTII component will be established by testing for the potency of a mutated LTII-derivative that lacks ganglioside binding, such as a LTIIa(T34I) mutation. The initial times for therapy administration post BoNT-intoxication will range from 1 h to one month, based upon a recent publication by our group that established primary rat spinal cord cells to detect long term BoNT/A intoxication. Therapies will be titered from 0.1 nM to 40 nM where a dose response for the delivery of cargo was observed for the βlac-LTII chimera in primary rat cortical neurons, and regeneration of SNAP25 will be monitored by Western blotting over time. This quantitation will allow subsequent modifications to the LTIIa delivery platform to optimize the translocation efficiency of the BoNT-LTIIa therapies. Note, the size of the most complex pan-serotype SNARE Binding Inhibitors are ˜30 kDa, while the α-LC Inhibitors to neutralize BoNT/A and BoNT/B are ˜40 kDa, showing the potential utility of developing the pan-serotype BoNT therapy, using SNARE Binding Inhibitors in tandem. Experiments will establish the potency to neutralize LC activity in enzymatic substrate cleavage reactions, adjustment to the therapies include adjusting the distances between the cargo and platform.

Anticipated Results. We anticipate that engineering the therapeutic-LTII chimeras will be straight forward, while optimizing expression and purification will require titration of culturing conditions and purification protocols. The therapeutic-LTIIs should assemble efficiently, based upon derivatives we have engineered and described in the preliminary results section. Potency of the α-LC camelid-LTIIa should be BoNT serotype specific. The advantage of the SNARE Binding Inhibitor therapy is the ability to modify the SNARE inhibitor to address the discovery of “new” BoNT serotypes. Addition of an E3 ubiquitin ligase F box should enhance the neutralization potency of the therapies, leading to faster therapeutic efficiency (FIG. 13). We do not anticipate that the platform will prove immune-stimulatory.

Alternative Approaches. Should the other camelid-LTII or the SNAP25-VAMP2-LTII derivatives have limited protection against BoNT challenge, experiments will establish the basis for the limited BoNT neutralization, measuring the potency to neutralize LC activity in an enzymatic reaction. This will also allow an estimation of the amounts of derivative needed to neutralize LC. These experiments will provide information on the rate limiting steps in the LC neutralization process. We anticipate that inserting the cargo at alternative sites along the A subunit may yield more efficient protein expression and/or assembly into and AB5 structure. Using structure-based prediction for the conservation of protein structure, several sites of cargo insertion will be tested should stability or assembly prove limiting for any of the cargo-LT chimeras engineered. The D4 α-LC/A camelid will also be tested relative to the B8-α-LC/A camelid-LTIIa that has neutralizing capacity. This analysis should provide new information of the neutralization mechanism of the camelid VHHs. The effect of the F box E3 ubiquitination ligase can only be assessed in cell models and not in vitro. Should LTIIa or cargo prove immune stimulatory, the epitopes that stimulate the immune response will be mapped as described by Pastan and coworkers who identified and eliminated the immune stimulatory epitopes within the binding domain of Pseudomonas aeruginosa exotoxin A (ETA) to facilitate the use of ETA as a therapeutic drug. Should the high affinity SNARE inhibitors prove potent therapies for BoNT/A and BoNT/B, protein modeling will predict interactions of other BoNT serotypes—SNARE substrate and introduce the mutations into the SNARE inhibitors. Protein therapies can also be added in trans to test potency verses versus fusion proteins. New therapies can be incorporated into LTII platforms as discovered.

This example will help to identify the optimal therapy that can be delivered into neurons by the LT platforms and establish the optimal stability and optimal efficiency for production for each LT-derivative.

### Example 5

### Determine the Potency of the BoNT Therapies in Mice −/+trans α-BoNT Antisera

The most potent neutralizing α-BoNT cargo-LTII therapies identified in cell culture experiments will be tested as a therapy in a mouse model of BoNT intoxication. This analysis will establish an average time to death curve (ATTD) using a standard preparation of BoNT/A. An IV method yields toxicity data in 30-70 min with concentrated BoNT solutions, while an IP method is completed in 3-4 days with more dilute BoNT solutions. We have also generated a rabbit pan-serotype specific α-BoNT-HCR(A-G) antisera (produced by Covance) that will be added in trans to determine if the trans-addition of α-BoNT antisera acts to lower toxin burden as a supplemental therapeutic strategy. This would indicate the presence of extracellular BoNT that enhances persistence of BoNT intoxication, in addition to the intracellular longevity of intracellular light chain.

Determine the potency of BoNT therapies in the average time to death curve (ATTD) model of botulism. The most potent neutralizing anti-BoNT cargo-LTIIa in the cell culture experiments will be tested as a therapy in a mouse model of BoNT intoxication, initially using BoNT/A and BoNT/B challenges. This analysis can establish an average time to death curve (ATTD) established from a standard preparation of BoNT/A. An IV method yields toxicity data in 30-70 min with concentrated BoNT solutions, while an IP method is completed in 3-4 days with more dilute BoNT solutions. Initially, BoNT-therapies will be administered with the BoNT challenge (10 LD₅₀) and then subsequent to BoNT challenge (starting at 1 h post challenge and extending this time with observed outcome data). Initial therapies will be administered at 40 nM, relative to the inoculum volume, and will be tittered up or down based upon the outcome of the initial challenge experiments. A detected protection by the BoNT-therapy will be followed by a challenge experiment using a heterologous BoNT serotype to test for “off-target” effects as previously described. Host response (pro-inflammatory immune cytokine production) and immunogenicity (stimulation of antibodies to LTIIa or cargo) to the BoNT therapies will be determined by collecting sera at 24 and 48 h post BoNT-therapeutic treatment.

Determine the potency of BoNT therapies in inhalation- and foodborne- models of BoNT intoxication. The optimal BoNT therapy identified above will also be evaluated in food- and inhalation- borne botulism in mouse challenge experiments. These challenge studies will use BoNT complexes, as these would be the most likely weaponized form of BoNTs, since these forms have increased stability and ease of production. BoNT/A complex has been shown to be significantly more toxic by the oral route than purified BoNT/A and no difference in toxin potency is observed for the inhalation route. In food-borne botulism, mice will be challenged with given a measured amount of BoNT delivered directly into the stomach by intragastric gavage. For the inhalation model, mice will be lightly anaesthetized with isoflurane and a 20 μl drop of BoNT (this model will tolerate up to 50 μl) will be placed into one nostril while the head of the animal remains in an upright position, until the BoNT is aspirated to minimize drainage as previously described. Both procedures are performed in a biosafety cabinet. For both routes of administration, BoNT dose will be determined for each serotype that causes death within 4 hours (˜50 LD50 Units of BoNT/A, 2 μg of toxin, will cause death within 4 h after IP injection). This will be the starting dose for challenge studies, followed by 10-fold increases if mice survive the initial challenge. Mice will be treated with BoNT therapy along with BoNT challenge or after BoNT challenge, at 1 h post challenge and extending to 2, 3, and 4 h post BoNT intoxication.

Anticipated Results. This example will establish the protective potency of the optimized β-BoNT therapy for protection in the average time to death curve (ATTD) model of botulism and then against inhalation botulism and foodborne botulism. We anticipate that the therapy will provide a high level of protection against each model of botulism, as the BoNTs exert their action after entry into the bloodstream, from which they distribute to the peripheral nervous system. While intranasal delivery of BoNTs as a model of inhalation botulism has previously been used, intranasal delivery does not perfectly mimic an aerosol exposure, which may result in greater toxicity.

Alternative approaches. The concentrations of BoNTs required to cause oral or inhalation botulism have only been described for BoNT/A in detail, and are significantly lower than by IP or IV injection. Thus, the corresponding concentrations for the other BoNT serotypes will be established within this study. Using oral gavage, sufficiently large volumes can be administered that a decrease in potency relative to BoNT/A will not be an obstacle. However, for the intranasal delivery, the maximum volume that can be delivered is 50 μl. Since our toxin purifications usually lead to highly concentrated toxin with typical concentrations in the range of 0.2-1 mg/ml, this will be sufficient for BoNT delivery that have an up to 50-fold reduced potency compared to BoNT/A. However, if a BoNT serotypes is even less potent by intranasal route, the protocol will be adjusted by lowering the challenge dose or giving repeat doses of toxin.

The present invention is not intended to be limited to the foregoing examples, but encompasses all such modifications and variations as come within the scope of the appended claims.

We claim:

1. A delivery platform comprising a toxin modified to incorporate a heterologous compound.

2. The platform of claim 1, wherein the toxin is an AB5 toxin.

3. The platform of claim 2, wherein the toxin has been modified to remove the A1 domain.

4. The platform of claim 2, wherein the toxin is selected from the group consisting to CT, LTI, LTIIa, LTIIb, LTIIC, and any recombinant LT derivative.

5. The platform of claim 1, wherein the toxin is LTIIa and the heterologous compound is β-lactamase.

6. The platform of claim 1, wherein the heterologous compound is selected from the group consisting of β-lactamase, a camelid antibody, a zinc-dependent protease, transcription factor EB (TFEB), an E3 binding protein, brain-derived neurotrophic factor (BDNF), a protease, a kinase, p53, phosphatase and tensin homolog (PTEN), Superoxide Dismutase 1 (SOD1), human hemochromatosis protein (HFE), caspase recruitment domain-containing protein 15 (CARD15), retinoblastoma gene product (pRB), and granulocyte-macrophage colony-stimulating factor (GM-CSF).

7. A method of treating botulism comprising administering a delivery platform comprising an AB5 toxin modified to incorporate β-lactamase to a subject in need thereof, wherein the botulism is treated.

8. The method of claim 7, wherein the AB5 toxin is LTIIa.

9. A method of treating Parkinson's Disease comprising administering a delivery platform comprising a toxin modified to incorporate a heterologous compound to a subject in need thereof, wherein the heterologous compound comprises a neuroprotective agent, and wherein the Parkinson's Disease is treated.

10. The method of claim 9, wherein the toxin is an AB5 toxin.

11. The method of claim 10, wherein the toxin has been modified to remove the A1 domain.

12. The method of claim 10, wherein the AB5 toxin is LTIIa.

13. The method of claim 9, wherein the neuroprotective agent is selected from the group consisting of BDNF, Brn4, and Progranulin.

14. A method of treating Cystic Fibrosis (CF) comprising administering a delivery platform comprising a toxin modified to incorporate a heterologous compound to a subject in need thereof, wherein the heterologous compound comprises a CFTR polypeptide, and wherein the CF is treated.

15. The method of claim 14, wherein the toxin is an AB5 toxin.

16. The method of claim 15, wherein the toxin has been modified to remove the A1 domain.

17. The method of claim 15, wherein the AB5 toxin is LTIIa.


SEQUENCE LISTING (9 sequences)

SEQ ID NO: 1 - protein, 6 aa, Escherichia coli
YIPGGR

SEQ ID NO: 2 - protein, 6 aa, Escherichia coli
RISRAK

SEQ ID NO: 3 - protein, 6 aa, Escherichia coli
INNNTD

SEQ ID NO: 4 - protein, 6 aa, Escherichia coli
VNTNTR

SEQ ID NO: 5 - protein, 10 aa, Escherichia coli
YIPGGRDYPD

SEQ ID NO: 6 - protein, 10 aa, Escherichia coli
RISRAKDYPD

SEQ ID NO: 7 - protein, 115 aa, Lama pacos
SGGGLVQPGGSLRLSCAASGSIFSIYAMGWYRQAPGKQRELVAAISSYGSTNYADSVKGRFTISRDNAKNTVYLQMNSLKPEDTAVYYCNADIATMTAVGGFDYWGQGTQVTVSS

SEQ ID NO: 8 - protein, 111 aa, Lama pacos
SGGGSVQPGGSLRLSCAAIGSVFTMYTTAWYRQTPGNLRELVASITDEHRTNYAASAEGRFTISRDNAKHTVDLQMTNLKPEDTAVYYCKLEHDLGYYDYWGQGTQVTVSS

SEQ ID NO: 9 - protein, 121 aa, Lama pacos
SGGGMVQPGGSLRLSCAASGFTFSTYDMSWVRQAPGKGPEWVSIINAGGGSTYYAASVKGRFAISRDNAKNTLYLQMNNLKPEDTALYYCARVASYYCRGYVCSPPEFDYWGQGTQVTVSS